SEQUENCE LISTING (34 sequences)

SEQ ID NO: 1 - DNA, 18 bp, Artificial Sequence
  M13 Primer Derivative
gtaaaacgacggccagtn

SEQ ID NO: 2 - DNA, 19 bp, Artificial Sequence
  M13 Primer Derivative
gtaaaacgacggccagtnn

SEQ ID NO: 3 - DNA, 20 bp, Artificial Sequence
  M13 Primer Derivative
gtaaaacgacggccagtnnn

SEQ ID NO: 4 - DNA, 21 bp, Artificial Sequence
  Synthetic Target
cgtaactggccgtcgttttac

SEQ ID NO: 5 - DNA, 19 bp, Artificial Sequence
  M13 Primer Derivative
gtaaaacgacggccagtna

SEQ ID NO: 6 - DNA, 20 bp, Artificial Sequence
  M13 Primer Derivative
gtaaaacgacggccagtnnc

SEQ ID NO: 7 - DNA, 21 bp, Artificial Sequence
  M13 Primer Derivative
gtaaaacgacggccagtnnng

SEQ ID NO: 8 - DNA, 19 bp, Artificial Sequence
  M13 Primer Derivative
gtaaaacgacggccagtng

SEQ ID NO: 9 - DNA, 19 bp, Artificial Sequence
  M13 Primer Derivative
gtaaaacgacggccagtnc

SEQ ID NO: 10 - DNA, 19 bp, Artificial Sequence
  M13 Primer Derivative
gtaaaacgacggccagtna

SEQ ID NO: 11 - DNA, 19 bp, Artificial Sequence
  M13 Primer Derivative
gtaaaacgacggccagtnt

SEQ ID NO: 12 - DNA, 20 bp, Artificial Sequence
  M13 Primer Derivative
gtaaaacgacggccagtnng

SEQ ID NO: 13 - DNA, 20 bp, Artificial Sequence
  M13 Primer Derivative
gtaaaacgacggccagtnnc

SEQ ID NO: 14 - DNA, 20 bp, Artificial Sequence
  M13 Primer Derivative
gtaaaacgacggccagtnna

SEQ ID NO: 15 - DNA, 20 bp, Artificial Sequence
  M13 Primer Derivative
gtaaaacgacggccagtnnt

SEQ ID NO: 16 - DNA, 20 bp, Artificial Sequence
  M13 Primer Derivative
gtaaaacgacggccagtnac

SEQ ID NO: 17 - DNA, 21 bp, Artificial Sequence
  M13 Primer Derivative
gtaaaacgacggccagtnncg

SEQ ID NO: 18 - DNA, 27 bp, Artificial Sequence
  M13 Primer Derivative
gtaaaacgacggccagtnnnngnnnnn

SEQ ID NO: 19 - DNA, 27 bp, Artificial Sequence
  M13 Primer Derivative
gtaaaacgacggccagtnnnncnnnnn

SEQ ID NO: 20 - DNA, 27 bp, Artificial Sequence
  M13 Primer Derivative
gtaaaacgacggccagtnnnnannnnn

SEQ ID NO: 21 - DNA, 27 bp, Artificial Sequence
  M13 Primer Derivative
gtaaaacgacggccagtnnnntnnnnn

SEQ ID NO: 22 - DNA, 27 bp, Artificial Sequence
  M13 Primer Derivative
gtaaaacgacggccagtnnnnngnnnn

SEQ ID NO: 23 - DNA, 27 bp, Artificial Sequence
  M13 Primer Derivative
gtaaaacgacggccagtnnnnncnnnn

SEQ ID NO: 24 - DNA, 27 bp, Artificial Sequence
  M13 Primer Derivative
gtaaaacgacggccagtnnnnnannnn

SEQ ID NO: 25 - DNA, 27 bp, Artificial Sequence
  M13 Primer Derivative
gtaaaacgacggccagtnnnnntnnnn

SEQ ID NO: 26 - DNA, 27 bp, Artificial Sequence
  Synthetic Target
cgtgatcgtaactggccgtcgttttac

SEQ ID NO: 27 - DNA, 21 bp, Artificial Sequence
  M13 Primer Derivative
gtaaaacgacggccagtnnnn

SEQ ID NO: 28 - DNA, 22 bp, Artificial Sequence
  M13 Primer Derivative
gtaaaacgacggccagtnnnnn

SEQ ID NO: 29 - DNA, 33 bp, Artificial Sequence
  Synthetic Target
gtatagcgtgatcgtaactggccgtcgttttac

SEQ ID NO: 30 - DNA, 23 bp, Artificial Sequence
  M13 Primer Derivative
gtaaaacgacggccagtnnnnnn

SEQ ID NO: 31 - DNA, 26 bp, Artificial Sequence
  M13 Primer Derivative
gtaaaacgacggccagtnnnnnnnnn

SEQ ID NO: 32 - DNA, 25 bp, Artificial Sequence
  M13 Primer Derivative
gtaaaacgacggccagtnnngatca

SEQ ID NO: 33 - DNA, 28 bp, Artificial Sequence
  M13 Primer Derivative
gtaaaacgacggccagtnnnnnncacgc

SEQ ID NO: 34 - DNA, 31 bp, Artificial Sequence
  M13 Primer Derivative
gtaaaacgacggccagtnnnnnnnnngctat

(19) United States
(12) Patent Application Publication
(10) Pub. No.: US 2003/0036073 A1
(43) Pub. Date: Feb. 20, 2003

(54) Matrix Sequencing: a novel method of polynucleotide analysis utilizing probes containing universal nucleotides

(76) Inventor: James Anthony Saba, Toledo, OH (US)

Correspondence Address: JAMES SABA, 2442 SECOR Rd., TOLEDO, OH, 43606, US

(21) Appl. No.: 10/156,144

(22) Filed: May 29, 2002

Related U.S. Application Data
(60) Provisional application No. 60/296,337, filed on Jun. 7, 2001.

(51) Int. Cl. C12Q 1/68; C07H 21/04; C12M 1/34

(52) U.S. Cl. 435/6; 435/287.2; 536/23.1

(57) ABSTRACT

Disclosed herein are materials and processes for a novel method of polynucleotide sequence analysis termed Matrix Sequencing. The invention utilizes a set of distinct probes, each distinct probe comprising a common first section (registering sequence) which specifically hybridizes to a target, and an adjoining second section consisting of universal nucleotides the number of which is distinct for each distinct probe. Microarrays of these novel probes, unlike those used in Sequencing by Hybridization (SBH), allow serial reading of the target sequence in a fashion similar to electrophoretic gels.

### CROSS-REFERENCE TO RELATED APPUCATIONS

This application claims the benefit of, and incorporates by reference, U.S. Provisional Application Ser. No. 60/296337 filed Jun. 7, 2001 and entitled “Nucleic Acids” by James Saba.

### BACKGROUND OF INVENTION

Nucleic acid sequence analysis is critical to the advancement of molecular biology, and there is considerable ongoing effort to make the process more efficient.

Relevant to the present invention is Head, et al (U.S. Pat. Nos. 6,322,968 & 6,337,188) which claim:

A sequencing reagent comprising one or more sequencing reagents wherein each reagent comprises:

i) a capture moiety which can form a stable complex with a region of a template nucleic acid molecule;

ii) a spacer region, and

iii) a sequence specific hybridizing region, wherein said sequence specific region comprises 4-8 bases which can hybridize to a complementary sequence on the template nucleic acid molecule.

The said spacer region preferably consists of a random sequence of nucleotides, and remains relatively constant. No mention is made of utilizing universal or degenerate nucleotides, or sets of probes whose spacers progressively increase in length. Further, their use of 4 to 8 terminal base-specific nucleotides is distinct from the present invention wherein there may be 1 to 3, if any. Drmanac (U.S. Pat. Nos. 6,270,961; 6,309,824 & 6,383,742); Ulfendahl (U.S. Pat. No. 6,280,954), Chetverin (U.S. Pat. No. 6,103,463) and Kambara (U.S. Pat. Nos. 5,741,644 & 5,935,794) teach arrayed probes containing a common target-hybridizing capture sequence which adjoins a second section of variable sequence and constant length.

Fugono (U.S. Pat. No. 5,738,993) teaches utilizing degenerate and universal nucleotides at the termini of probes to modify their hybridization stringency.

Preparata et al (U.S. patent application Ser. No. 20010004728) teach “gapped” sequencing probe sets which include any repeating pattern of universal (U) and designate (X) nucleotides, e.g., UUXUXXUX. Preferably the probes are iterative, e.g., UUXXUUXXUUXX, UXUXUXUX.

Almost a decade ago, Nichols, et al (Nature 1994 June 9;369(6480):492-3) synthesized a universal nucleotide which, when placed near or even at the 3′ end of a primer, did not preclude primer extension.

These articles (incorporated in their entirety by reference) are valuable in defining the prior art, and their experimental methodology is often applicable to the present invention.

### SUMMARY OF INVENTION

Disclosed herein are materials and processes for a novel method of polynucleotide sequence analysis termed Matrix Sequencing. The invention utilizes a set of distinct probes, each distinct probe comprising a common first section (registering sequence) which specifically hybridizes to a target, and an adjoining second section consisting of universal nucleotides the number of which is distinct for each distinct probe. Microarrays of these novel probes, unlike those used in Sequencing by Hybridization (SBH), allow serial reading of the target sequence in a fashion similar to electrophoratic gels.

### BRIEF DESCRIPTION OF DRAWINGS

FIG. 1. Arrayed probes with incrementally increasing lengths of universal nucleotide-containing second sections, and whereby the sequencing is achieved by primer extension utilizing base-specifically labeled chain terminating nucleotides.

FIG. 2. Derivation wherein the arrayed probes have one base-specific nucleotide at their 3′ end which interrogates a specific target nucleotide.

FIG. 3. Derivation or FIG. 2, termed Scanning Mismatch Sequencing, where the probes are equivalent in length due to additional universal nucleotides following the interrogating nucleotide.

FIG. 4. Herein target-hybridized probes are extended by ligation of distinctly labeled oligonucleotides.

FIG. 5. Novel labeling scheme with potential utility for labeling the large number of distinctly labeled oligonucleotides needed in the process exemplified by FIG. 4.

### DETAILED DESCRIPTION OF INVENTION

A “nucleotide” denotes a polynucleotide monomer which resides in, or has the potential to reside in a polynucleotide. There are a myriad of known and synthetically feasible nucleotide derivations.

A “universal nucleotide” can match up (“base-pair”) with the naturally occurring nucleotides with similar tenacity (1-13).

A “degenerate nucleotide” can base-pair with multiple but not all of the four naturally occurring nucleotide groups (adenosines, guanosines, cytidines, or thymidines/uridines).

A “base-specific nucleotide” can efficiently base-pair to oily one of the four naturally occurring nucleotide groups.

A “probe” comprises a polynucleotide. In certain processes the probe functions as a primer.

Probes are preferably covalently or noncovalently affixed, via their 5′ or 3′ termini, to a support(s) prior to or after target hybridization. Supports can be of various configurations, composed of various materials, and include soluble polyvalent polymers. Preferably the support is a chip wherein distinct probes are arrayed at unique locations (14-20). Coded beads are also applicable (21-24).

A “Target” is a polynucleotide, most commonly DNA or RNA.

An entity is considered “distinct” when in some intrinsic characteristic it is different from others. An unqualified statement such as “probes” or “targets” optionally indicates multiple identical or distinct entities.

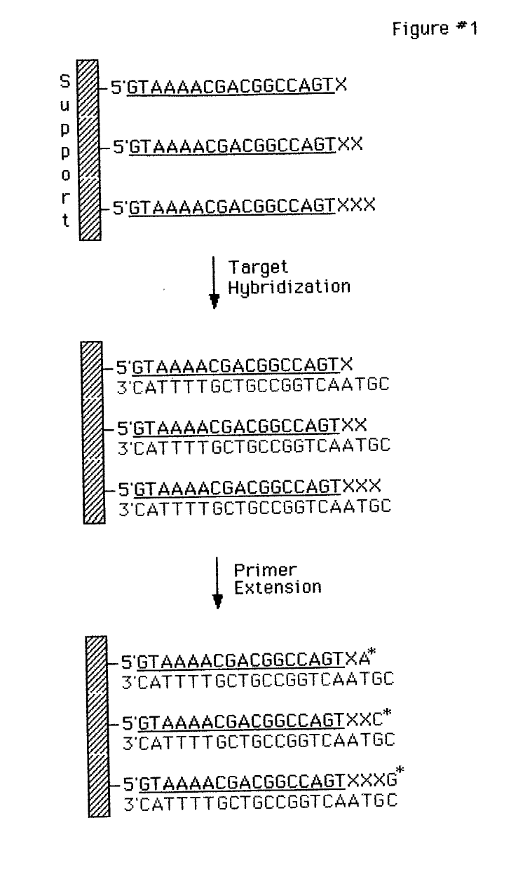

As exemplified in FIG. 1, the novel probes of the present invention comprise two adjoining sections. The first probe section termed “registering sequence” (herein the M13 Universal Primer) is proximal the support and specifically hybridizes to the target. Each distinct probe is affixed to the support at a unique position, and in reality there are many identical probes at each position.

Registering sequences are preferably 4 or more nucleotides in length. The lengths of the universal nucleotide-containing second sections are limited only by their ability to appropriately hybridize to the targets. Note the potential for multiplex sequencing of distinct targets, wherein multiple probe sets having distinct registering sequences are simultaneously utilized.

In the first step of FIG. 1 the registering sequences are specifically hybridized to the targets so as to precisely align the hybridization of the incrementally increasing universal nucleotide (“X”)-containing second sections. Of course the probe composition and the hybridization conditions should be such that probe-target hybridizations are as required. Nucleotide derivations can profoundly affect the specificity and efficiency of hybridizations. Also, diverse reagents and various proteins may aid in achieving precise probe-target hybridizations (26-36). Numerous computer programs and schemes for selection of optimal hybridizing sequences are available (37-40). Potentially problematic are unintentional hybridizations by the universal nucleotide-containing second sections (8, 41), and preferably these sections hybridize with less stringency than the registering sequences. Conditions could even be devised whereby hybridization of probes to targets is accomplished in two stages; a first stringent stage where only the registering sequences hybridize, followed by the lowering of stringency to allow hybridization by the universal nucleotide-containing second sections. One interesting means by which this might be accomplished is by controlling hybridizations electronically (42-44). Conceivably the probes and targets could be designed so that if a probe is not appropriately hybridized to a target, it can be disabled in its capacity to be labeled, such as by enzymatic hydrolysis.

Continuing with FIG. 1, subsequent to precise hybridization of probe to target, the probe is extended by one fluorescently labeled (“*”) chain terminating nucleotide, the identity of which is specified by the target sequence (45-53). It is of course important that the particular reaction conditions, polymerase, and terminating nucleotides utilized are such that the presence of the universal nucleotides does not preclude extension (1-2, 54, 55). A large number of other labeling and detection schemes are applicable. Particularly, electronic biochips for detection are attracting considerable attention (56-63).

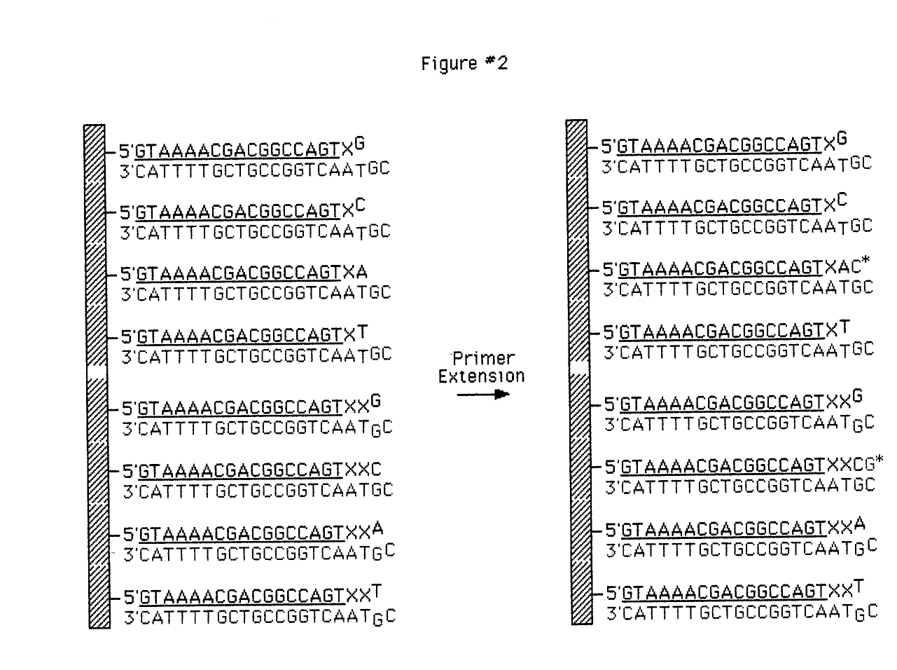

The derivation exemplified in FIG. 2 is similar to that in FIG. 1 except that each probe has one base-specific nucleotide at their 3′ end which interrogates a specific target nucleotide. Unlike FIG. 1, each target nucleotide being identified requires a subset of four probes rather than one. The probe of each subset that this interrogating nucleotide correctly base-pairs with the target is selectively extended by polymerase incorporation of a labeled nucleotide. Conceivably, the probes in this example could have 2 or even 3 terminal base-specific nucleotides interrogating the target sequence. Note the redundancy of sequence information due to the probes identifying overlapping dinucleotides; and the potential to increase the incremental steps from 1 to 2 universal nucleotides.

An alternative to the process in FIG. 2 would be to initially have each probe's 3′ terminal nucleotide labeled. After target hybridization, those labeled terminal nucleotides which are mismatched could be selectively removed, such as by an error correcting polymerase.

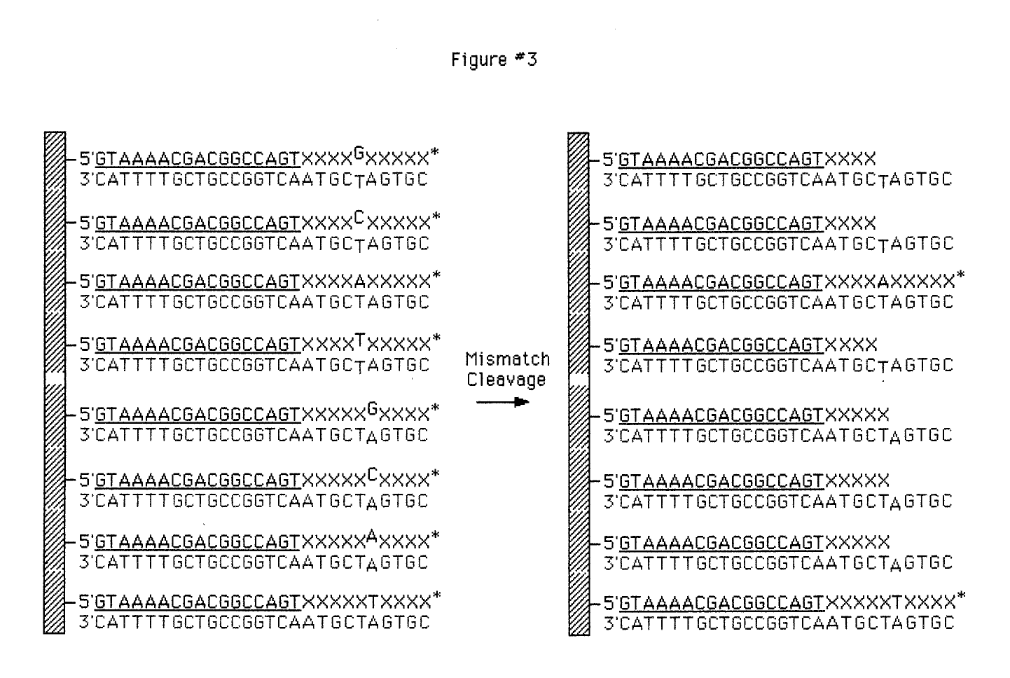

Another alternative to FIG. 2 is shown in FIG. 3. In this derivation termed Scanning Mismatch Sequencing the the probes are equivalent in length due to additional universal nucleotides following the interrogating nucleotide. This may aid in more uniform probe-target hybridizations, and expands the potentially useful labeling and detection schemes. In this example mismatched probes are detected by their selective cleavage and concurrent loss of prelabeled 3′ ends (64-70).

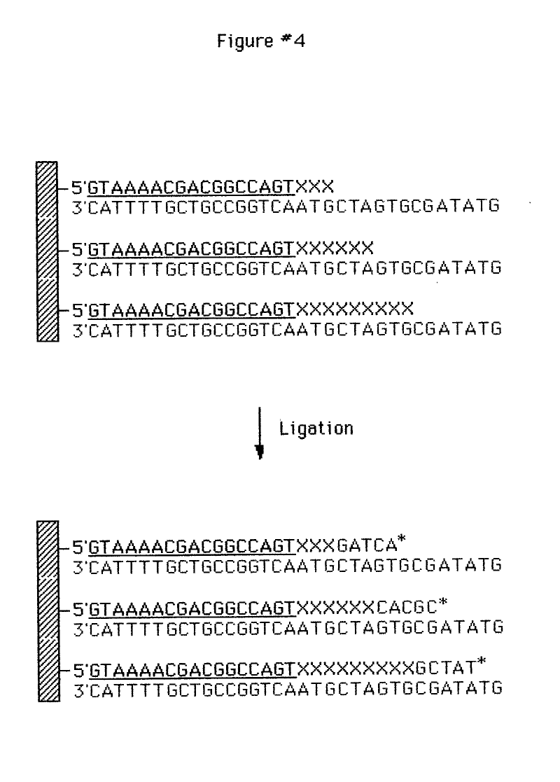

FIG. 4 exemplifies a notably distinct derivation, wherein the hybridized probes are ligated to labeled oligonucleotides as directed by the target. Most importantly, the incremental increases in the lengths of the universal nucleotide-containing second sections of the probes can be more than 1 nucleotide; thus offering the possibility of considerably reducing the number of distinct probes required to sequence a given target. Also note (as shown) if the incremental increases are smaller than the length of the ligated oligonucleotides, then there is an overlap of sequences read and thus greater accuracy. As in prior figures, it may be advantageous to use subsets of probes which have 1-3 base-specific and/or degenerate nucleotides at their distal termini. The termini of the labeled oligonucleotides not intended to be ligated to the probes may be such as to prevent multiple ligations of adjoining (stacked) oligonucleotides (71, 72). Ligation is preferably achieved enzymatically, yet it can also be achieved chemically or by radiation.

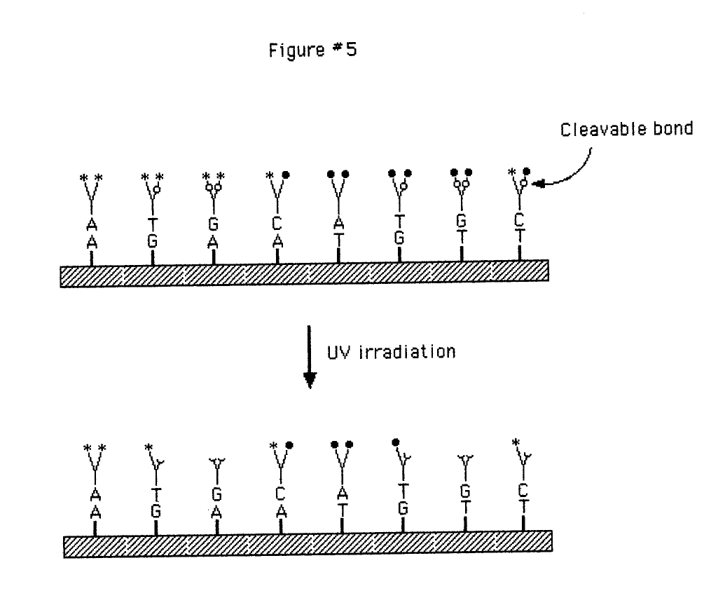

Labeling the required large number of distinct oligonucleotides is preferably via mass spectrometry labels (73). A potential alternative is exemplified in FIG. 5. In this very rudimentary example we are determining the identities of 8 arrayed dinucleotides. The labeling of each dinucleotide is prior knowledge and consists of two labels, which are selected from a group of two distinct labels (“*” & “●”). Some of these labels are conjugated to a dinucleotide via a UV labile bond (“o”) which allows selective liberation of these labels (76-79). The dinucleotides are easily identified by simple comparison of the quantitative or qualitative signals before and after irradiation.

In general the labeling scheme involves a multiply labeled entity, and a subsequent step wherein a subset of these labels is selectively liberated, disabled or enabled. The disabling or enabling occur by the making and/or breaking of chemical bonds, and an example thereof would be the bleaching of a fluorescent dye.

The term “labels” as used here is quite broad in that it includes not only those substances which emit or can be induced to emit signals, but also includes substances which can appreciably alter the signals of an adjacent label. Good examples of labels are fluorescent dyes, fluorescent energy transferers, fluorescent quenchers.

These examples and accompanying figures have deliberately been made exceptionally simple so as to clearly and concisely present the invention. Further information can be found in the accompanying U.S. Provision Patent Application No. 60/296337. Many modifications and variations of the present invention are possible, and it is intended that all such modifications and variations be included within the scope of present invention as defined by the claims.

### REFERENCES

The following articles are incorporated in their entirety by reference. They more fully describe the state of the art, and teach applicable material and methods.

1) A universal nucleoside for use at ambiguous sites in DNA primers. Nichols, et al Nature 1994 June 9;369(6480):492-3

2) Synthesis, Structure, and Deoxyribonucleic Acid Sequencing with a Universal Nucleoside: 1-(2′-Deoxy-BD-ribofuranosyl)-3-nitropyrrole. Bergstrom, et al American Chemical Society 1995 177(4):1201-1209.

3) Survey and summary: The applications of universal DNA base analogs. Loakes, D Nucleic Acids Res May 21, 2001; 29(12):2437-2447

4) Universal bases for hybridization, replication and chain termination. Berger, et al Nucleic Acids Res Aug. 1, 2000;28(15):2911-2914

5) Peptide nucleic acid-DNA duplexes containing the universal base 3-nitropyrrole. Zhang, et al Methods 2001 February;23(2):132-40

6) Effect of the universal base 3-nitropyrrole on the selectivity of neighboring natural bases. Oliver, et al Org Lett 2001 June 28;3(13):1977-80

7) Significance of nucleobase shape complementarity and hydrogen bonding in the formation and stability of the closed polymerase-DNA complex. Dzantiev, et al Biochemistry Mar. 13, 2001;40(10):3215-21

8) Melting studies of short DNA hairpins containing the universal base 5-nitroindole. Vallone, et al Nucleic Acids Res Sep. 1, 1999;27(17):3589-96

9) Polymerase recognition of synthetic oligodeoxyribonucleotides incorporating degenerate pyrimidine and purine bases. Hill, et al Proc Natl Acad Sci USA Apr. 14, 1998;95(8):4258-63

10) Oligonucleotides having universal nucleoside spacers. Bergstrom, et al U.S. Pat. No. 5,681,947 October 1997

11) Nucleotide analogs and new buffers improve a generalized method to enrich for low abundance mutations Day, et al Nucleic Acids Res 1999 27(8):1819-1827

12) Improved fidelity of thermostable ligases for detection of microsatellite repeat sequences using nucleoside analogs. Zirvi, et al Nucleic Acids Res 1999 27(24):e41

13) Synthesis of pyrrole carboxamide nucleotide triphosphates-putative labelled nucleotide analogues. Nairne, et al Tetrahedron Letters 2002, 43:12:2289-2291

14) Oligonucleotides Fodor, et al U.S. patent application Ser. No. 20010053519 December 2001

15) High density synthetic oligonucleotide arrays (see supplement for other relevant articles). Lipshutz, et al Nat Genet 1999 January;21(1 Suppl):20-4

16) Molecular Interactions on Microarrays (see suppliment for other relevant articles) Southern, et al Nature Genetics Suppl. Jan. 21, 1999:5-9

17) Parallel genotyping of human SNPs using generic high-density oligonucleotide tag arrays Fan, et al Genome Res 2000 June;10(6):853-60

18) Detection of specific alleles by using allele-specific primer extension followed by capture on solid support. Ugozzoli, et al Genet Anal Tech Appl 1992 August;9(4):107-12

19) Universal DNA microarray method for multiplex detection of low abundance point mutations. Gerry, et al J Mol Biol Sep. 17, 1999;292(2):251-62

20) Microfabricated, flowthrough porous apparatus for discrete detection of binding reactions. Beattie, et al U.S. Pat. No. 5,843,767 December 1998

21) DNA hybridization on microparticles: determining capture-probe density and equilibrium dissociation constants. Wilkins, et al Nucleic Acids Res Apr. 1, 1999;27(7):1719-27

22) Method of identifying nucleotide differences. Fodor, et al U.S. Pat. No. 5,925,525 July 1999

23) Multiplexed particle-based flow cytomatric assays (review). Vignali, D A J Immunol Methods Sep. 21. 2000;243(1-2):243-55

24) A Bead-Based Method for Multiplexed Identification and Quantitation of DNA Sequences Using Flow Cytometry. Spiro, et al Appl Environ Microbiol 2000 October;66(10):4258-4265

26) Compositions and methods for enhancing hybridization and priming specificity. Van Ness, et al U.S. Pat. No. 6,361,940 March 2002

27) Methods and compositions for modulating melting temperatures of nucleic acids. Lane, et al U.S. Pat. No. 6,221,589 April 2001

28) Arrays of modified nucleic acid probes and methods of use. McGall, et al U.S. Pat. No. 6,156,501 December 2000

29) Rapid assembly and disassembly of complementary DNA strands through an equilibrium intermediate state mediated by A1 hnRNP protein. Pontius, et al J Biol Chem Jul. 15, 1992;267(20):13815-8

30) DNA strand exchange and selective DNA annealing promoted by the HIV-1 nucleocapsid protein. Tsuchihashi, et al J Virol 1994 September;68(9):5863-70

31) Annealing of complementary DNA strands above the melting point of the duplex promoted by an archaeal protein. Guagliardi, et al J Mol Biol Apr. 11, 1997;267(4):841-8

32) Rapid renaturation of complementary DNA strands mediated by cationic detergents: a role for high-probability binding domains in enhancing the kinetics of molecular assembly processes. Pontius, et al Proc Natl Acad Sci USA Sep. 15, 1991;88(18):8237-41

33) Minimising the secondary structure of DNA targets by incorporation of a modified deoxynucleoside: implications for nucleic acid analysis by hybridisation. Nguyen, et al Nucleic Acids Res Oct. 15, 2000;28(20):3904-9

34) The tetramethylammonium chloride method for screening of cDNA libraries using highly degenerate oligonucleotides obtained by backtranslation of amino-acid sequences. Honore, et al J Biochem Biophys Methods 1993 August;27(1):39-48

35) Nucleic acid chaperone activity of the ORF1 protein from the mouse LINE-1 retrotransposon. Martin, et al Mol Cell Biol 2001 January;21(2):467-75

36) Method for nucleic acid hybridization using single-stranded DNA binding protein. Tabor, et al U.S. Pat. No. 5,534,407 July 1996

37) Octamer-primed sequencing technology: development of primer identification software. Mei, et al Nucleic Acids Res Apr. 1, 2000;28(7):E22

38) Method of synthesizing diverse collections of oligomers. Dower, et al U.S. Pat. No. 6,143,497 November 2000

39) Method, apparatus and computer program product for determining a set of non-hybridizing oligonucleotides. Brenner, S U.S. Pat. No. 6,138,077 October 2000

40) Optimized primer library for gene sequencing and method of using same. Hardin, et al U.S. Pat. No. 6,083,695 July 2000

41) Nucleic acid hairpin probes and uses thereof. Dattagupta U.S. Pat. No. 6,380,377 April 2002

42) Rapid, high fidelity analysis of simple sequence repeats on an electronically active DNA microchip. Radtkey, et al Nucleic Acids Res Apr. 1, 2000;28(7):E17

43) Methods for electronic stringency control for molecular biological analysis and diagnostics Heller, M J U.S. Pat. No. 6,017,696 January 2000

44) Electric field amplified oligonucleotide ligase analysis. Shieh, et al U.S. Pat. No. 6,030,781 February 2000

45) Method for determining specific nucleotide variations by primer extension in the presence of mixture of labeled nucleotides and terminators. Soderlund, et al U.S. Pat. No. 6,013,431 January 2000

46) Method for the detection of genetic diseases and gene sequence variations by single nucleotide primer extension. Bajaj, et al U.S. Pat. No. 5,846,710 December 1998

47) From gels to chips: “minisequencing” primer extension for analysis of point mutations and single nucleotide polymorphisms (See references therein). Syvanen, A C Hum Mutat. 1999;13(1):1-10

48) A microsphere-based assay for multiplexed single nucleotide polymorphism analysis using single base chain extension. Chen, et al Genome Res. 2000 April;10(4):549-57

49) Primer specific and mispair extension analysis (PSMEA) as a simple approach to fast genotyping. Hu, et al Nucleic Acids Res Nov. 1, 1998;26(21):5013-5

50) Parallel primer extension approach to nucleic acid sequence analysis. Caskey, et al U.S. Pat. No. 6,153,379 November 2000

51) Arrayed primer extension: solid-phase four-color DNA resequencing and mutation detection technology. Kurg, et al Genet Test. 2000;4(1):1-7

52) Polymorphism analysis and gene detection by minisequencing on an array of gel-immobilized primers. Dubiley, et al Nucleic Acids Res Sep. 15, 1999;27(18):e19

53) Single-nucleotide polymorphism analysis by MALDI-TOF mass spectrometry (Review). Griffin & Smith Trends in Biotechnology 2000;18:77-84

54) Effect of manganese ions on the incorporation of dideoxynucleotides by bacteriophage T7 DNA polymerase and Escherichia coli DNA polymerase I. Tabor, et al PNAS 1989 86(11):4076-80

55) A single residue in DNA polymerases of the Escherichia coli DNA polymerase I family is critical for distinguishing between deoxy- and dideoxyribonucleotides. Tabor, et al Proc Natl Acad Sci USA Jul. 3, 1995;92(14):6339-43

56) Highly sensitive biological agent probe. Megerle U.S. Pat. No. 6,391,624 May 2002

57) Methods for determination of single nucleic acid polymorphisms using bioelectronic microchip. Nerenberg, et al U.S. patent application Ser. No. 20010014449 August 2001

58) Electrochemical sensor using intercalative, redox-active moieties. U.S. patent application Ser. No. 20020055103 Barton, et al May 2002

59) Mutation detection by electrocatalysis at DNA-modified electrodes. Boon, et al Nat Biotechnol 2000 October;18(10):1096-100

60) Single-base mismatch detection based on charge transduction through DNA. Kelley, et al Nucleic Acids Res Dec. 15, 1999;27(24):4830-7

61) M-DNA: A complex between divalent metal ions and DNA which behaves as a molecular wire. Aich, et al J Mol Biol Nov. 26, 1999;294(2):477-85

62) Methods and apparatus for the photo-electrochemical detection of nucleic acid. Netzel, T U.S. Pat. No. 6,180,350 January 2001

63) Nucleic acid mediated electron transfer. Meade, et al U.S. Pat. No. 6,177,250 January 2001

64) Enzymatic and chemical cleavage methods. (Review)” Taylor, G R Electrophoresis 1999 June;20(6):1125-30

65) A versatile mismatch recognition agent: specific cleavage of a plasmid DNA at a single base mispair. Jackson, et al Biochemistry Apr. 13, 1999;38(15):4655-62

66) Detection of 81 of 81 known mouse beta-globin promoter mutations with T4 endonuclease VII—the EMC method. Youil, et al Genomics Mar. 15, 1996;32(3):431-5

67) Enzymatic mutation detection. Phosphate ions increase incision efficiency of endonuclease VII at a variety of damage sites in DNA. Golz, et al Mutat Res 1998 May;382(3-4):85-92

68) Reactivity of potassium permanganate and tetraethylammonium chloride with mismatched bases and a simple mutation detection protocol. Lambrinakos, et al Nucleic Acids Res Apr. 15, 1999;27(8):1866-74

69) Evaluation of MutS as a tool for direct measurement of point mutations in genomic DNA. Parsons, et al Mutat Res Mar. 21, 1997;374(2):277-85

70) One tube mutation detection using sensitive fluorescent dyeing of MutS protected DNA. Sachadyn et al Nucleic Acids Res Apr. 15, 2000;28(8):E36

71) DNA sequencing by hybridization to microchip octa- and decanucleotides extended by stacked pentanucleotides. Parinov, et al Nucleic Acids Res Aug. 1, 1996;24(15):2998-3004

72) Mutation detection by ligation to complete n-mer DNA arrays. Gunderson, et al Genome Res 1998 November;8(11):1142-53

73) Tag reagent and assay method. Southern, et al U.S. Pat. No. 5,770,367 June 1998

74) Method for sequencing polynucleotides. Cheeseman, P U.S. Pat. No. 5,302,509 April 1994

75) DNA sequencing by parallel oligonucleotide extensions. Macevicz, S C U.S. Pat. No. 5,750,341 May 1998

76) Photocleavable agents and conjugates for the detection and isolation of biomolecules” Rothschild, et al U.S. Pat. No. 6,057,096 May 2000 (also see U.S. Pat. Nos. 5,986,076 & 5,948,624)

77) DNA probes using fluorescence resonance energy transfer (FRET): designs and applications. Didenko, V Biotechniques 2001 November;31(5):1106-16, 1118, 1120-1

78) Design and synthesis of fluorescence energy transfer dye-labeled primers and their application for DNA sequencing and analysis. Ju, et al Anal Biochem Oct. 10, 1995;231(1):131-40

79) Pyrimidines linked to a quencher. Nardone, et al U.S. Pat. No. 6,117,986 September 2000

1) a set of distinct polynucleotide probes, each distinct probe comprising:
i) a common first section (registering sequence) which specifically hybridizes to a target;
ii) an adjoining second section consisting of universal nucleotides, the number of which is distinct for each distinct probe; and
iii) optionally 1-3 base-specific and/or degenerate nucleotides linked to the free terminus of the second section.

2) The set of distinct probes of claim 1 arrayed on a support.

3) The arrayed probes of claim 2 wherein the registering sequence is proximal the support, and the free termini of the universal nucleotide-containing second sections are each linked to 1 base-specific and/or degenerate nucleotide.

4) The arrayed probes of claim 2 wherein the registering sequence is proximal the support, and the free termini of the universal nucleotide-containing second sections are not linked to a base-specific or degenerate nucleotide.

5) A process of polynucleotide sequence analysis comprising hybridizing targets to the set of distinct probes of claim 1.

6) A process of polynucleotide sequence analysis comprising hybridizing targets to the arrayed set of distinct probes of claim 2.

7) The process of claim 6 wherein subsequent to hybridizations, a mixture of labeled nucleotides is provided.

8) The process of claim 6 wherein subsequent to hybridizations, a mixture of labeled oligonucleotides is provided.

9) A multiply labeled entity wherein a subset of the labels can be selectively liberated, disabled, or enabled such that the signaling from the entity is altered.

10) An array of distinct, multiply labeled entities as defined by claim 9.

11) A multiply labeled entity wherein a subset of the labels can be selectively liberated such that the signaling from the entity is altered.

12) A process of identifying the multiply labeled entity of claim 9 comprising:
i) detecting the signals (or lack thereof) from the labeled entity;
ii) subjecting the labeled entity to a process whereby a subset of the labels is selectively liberated, disabled, or enabled such that the signaling from the entity is altered.
iii) detecting the new signals (or lack thereof) and making a comparison with the signals (or lack thereof) obtained in step (i).
